(19) United States
(12) Patent Application Publication
(10) Pub. No.: US 2020/0016563 A1
(43) Pub. Date: Jan. 16, 2020

(54) POLYURETHANE MICROCARRIER AND PREPARATION METHOD AND USE THEREOF

(71) Applicant: WEST CHINA HOSPTIAL, SICHUAN UNIVERSITY, Chengdu, Sichuan (CN)

(72) Inventors: Huiqi XIE, Chengdu, Sichuan (CN); Li DONG, Chengdu, Sichuan (CN); Mei GONG, Chengdu, Sichuan (CN); Anjing CHEN, Chengdu, Sichuan (CN)

(21) Appl. No.: 16/476,211

(22) PCT Filed: Dec. 20, 2017
(86) PCT No.: PCT/CN2017/117530

§ 371 Date: Oct. 3, 2019

(30) Foreign Application Priority Data
Jan. 6, 2017 (CN) 201710010320.9

(51) Int. Cl. B01J 13/14; A61K 47/34; C08G 18/75; A61L 27/50; C08G 8/10; C08G 18/12; C08G 18/32; C08G 18/42; C08G 18/48; A61L 27/18

(52) U.S. Cl. CPC B01J 13/14; A61K 47/34; C08G 18/755; A61L 27/50; C08G 8/10; A61L 2300/258; C08G 18/3206; C08G 18/4277; C08G 18/4833; A61L 27/18; C08G 18/12

(57) ABSTRACT

The present invention discloses a polyurethane microsphere with diameter around 150 μm-270 μm, as well as the preparation method and use of it. The polyurethane which is prepared by this method has good biocompatibility, and it can be used as microcarrier to enhance cell proliferation. Meanwhile, the polyurethane microsphere is also injectable and enables to be used in tissue repair, evidently showing a well clinical application prospect.

### TECHNICAL FIELD

The present invention involves in a polyurethane microcarrier, as well as the preparative and uses thereof, and it belongs to the biomaterial field.

### BACKGROUND

Microcarrier is a kind of bead with diameter around 60-300 μm which is suitable for anchorage-dependent cells attachment and growth on. Microcarrier offers a series of advantages for cell expansion: Microcarrier provides requisite surface for the adhesion and proliferation of anchorage-dependent cells; Due to the large surface/volume ratio, microcarrier offers an amplified homogeneous cultural system in finite space; After being clustered with cells on the surface microcarriers and cells can form cell-microcarriers complexes, which promote the interaction among cells, and the secretion of cells support the intrcellular activity further.

Existing microcarriers are usually used for the culture of cells in vitro. With the development of bio-medical materials, cells-laden microcarrier shows increasingly advantages for tissue engineering strategy. Microcarriers serve as cell delivery system not only enhance the proliferation of cells, but also avoid the cells mortality and dispersion caused by mere cell-injection.

Injectable microcarriers can repair tissue defect via minimal invasion, which avoid wound caused by surgery intervention. Injection of cell-microcarrier complexes is a fairly straightforward application for rapid tissue regeneration, and this method has already been widely researched in tissue engeering. At present, gelatin-based microcarriers are most used in large-scale expansion of cells. Although they exhibited great promotion of cells proliferation, the poor mechanical property and the biodegradability of the natural polymers limite their application in tissue repair.

Compared with natural microcarriers, the synthetic microcarriers have incomparable superiority in mechanical property. However, it's hard to satisfy simultaneously the culture cells in high efficiency and injectability. Take polyurethane for instance, the reported polyurethane used as carrier device is polyurethane foam which cannot be transported via injection, and therefore the applications in tissue repair is limited.

### CONTENT OF THE INVENTION

In order to resolve above problems, the present invention provides a novel polyurethane microcarrier, i.e. polyurethane microsphere as well as the preparation and uses thereof.

For polyurethane microsphere of the present invention, its diameter is 150 μm-270 μm.

Wherein, said polyurethane microsphere is prepared according to the following method:

(1) Two kinds of oligodiols are premixed;

### (2) Pre-polymerization:

Isocyanate and oligodiols in step (1) are starting materials, and they are added to the reaction vessel and stirred;

### (3) Chain-extension:

After above step (2), hydrophilic chain extender is added, and at the same time, the temperature is reduced, and the reactant is stirred;

### (4) Neutralization:

Neutralizer is added, and the reactant is continually stirred;

### (5) Emulsification:

Under stirring, the polyurethane synthesized in step (4) is added dropwise to the distilled water and dispersed;

(6) The polyurethane microsphere with particle diameter of 150 μm-270 μm is purified, sieved, and collected.

The present invention further provides the method of above-mentioned polyurethane microsphere, with the following steps:

(1) Two kinds of oligodiols are premixed;

### (2) Pre-polymerization:

Isocyanate and oligodiols in step (1) are starting materials, and they are added to the reaction vessel and stirred;

### (3) Chain-extension:

After above step (2), hydrophilic chain extender is added, and at the same time, the temperature is reduced, and the reaction is stirred;

### (4) Neutralization:

Add neutralizer and continue with stirring;

### (5) Emulsification:

Under stirring, the polyurethane synthesized in step (4) is added dropwise to the distilled water and dispersed;

(6) The polyurethane microsphere with particle diameter of 150 μm-270 μm is purified, sieved, and collected.

Preferably, in step (1), two kinds of different oligodiols used in step (1) are optionally selected from the group of polyethylene glycol, poly(caprolactone)diol, and polytetrahydrofuran; preferably, two kinds of different oligodiols used in step (1) are polyethylene glycol and poly(caprolactone)diol or polytetrahydrofuran;

Further, said poly(caprolactone)diol is poly(caprolactone)diol2000 and/or said polyethylene glycol is polyethylene glycol200;

Further, in step (1), the molar ratio of poly(caprolactone)diol and polyethylene glycol is 1:1-2:1;

Further, in step (1), the molar ratio of polytetrahydrofuran and polyethylene glycol is 1:1-2:1;

Preferably, in step (2), the molar ratio of isocyanate and the total oligodiols in step (1) is (2-3):1, preferably 3:1;

and/or in step (2), said isocyanate is optionally selected from the group of isophorone diisocyanate, L-lysine diisocyanate, and diphenylmethane diisocyanate; preferably, said isocyanate is isophorone diisocyanate;
and/or in step (2), for stirring at speed of 350-700 rpm, preferably, the stirring speed is 380 rpm; the reaction time is 2-4 hours, preferably 2.5 h.

Preferably, in step (3), the molar ratio of said chain extender and isocyanate in step (2) is (0.1-1):(1), preferably 0.5:1;

and/or in step (3), said chain extender is 2,2-dihydroxymethylbutyric acid or 2,2-dihydroxymethylpropionic acid; preferably, said chain extender is 2,2-dihydroxymethylbutyric acid;
and/or in step (3), said reducing the temperature means the temperature is reduced to 45-55° C., preferably 50° C.; said stirring is carried out at the speed of 350-700 rpm, and preferably the stirring speed is 380 rpm; the reaction time is 1-3 hours, preferably 1.5 h.

Preferably, in step (4), the neutralizer and the chain extender in step (3) are equimolar; and/or in step (4), said neutralizer is triethylamine or sodium hydroxide;

and/or in step (4), said stirring is carried out at the speed of 350-700 rpm, preferably the stirring speed is 380 rpm; the reaction time is 15 min.

Preferably, in step (5), said stirring speed is 350-700 rpm, preferably 500 rpm.

Preferably, the method in step (6) is: the polyurethane particles obtained by reaction of step (5) are washed with the distilled water, dried in vacuum to the constant weight, and sieved with 50-100 meshes to select the microspheres with particle diameter of 150-270 μm.

The present invention also provides the use of polyurethane microsphere above-mentioned in the preparation of microcarrier materials.

The present invention also provides a materiel for tissue repair in vivo characterized in that which is prepared by combining the polyurethane microsphere as microcarrier with cells.

The polyurethane microcarrier of the present invention has the following beneficial effects:

The polyurethane microcarrier of the present invention has good biocompatibility, and can support growth of adherent cells;

The present invention can optimize the diameter of polyurethane microcarrier to fit for the adherence and expansion of cells on its surface, and the particle size is uniform and controllable, that breaks the application limitation of polyurethane carrier as a drug carrier;

In the preparation process of polyurethane microsphere of the present invention, organic medium with high boiling point are not needed and it is non-cytotoxicity, and has low environmental impact;

In the suspension culture process, polyurethane microcarrier according to the present invention is dispersed and would be non-aggregation, that ensures the valid size for injection;

The polyurethane microcarrier system according to the present invention can realize high-yield cells proliferation in the finite space;

The polyurethane microcarrier according to the present invention has a low cost, and can be recycled.

To sum up, the polyurethane which is prepared by this method has good biocompatibility, and it can be used as microcarrier and enhances cell proliferation. Meanwhile, the polyurethane microsphere is also injectable and enables to be used in tissue repair, evidently showing a well clinical application prospect.

In the following, the present invention is further illustrated by referring to the specific examples, but the present invention is not limited. Without departing from above basic technical spirit of the present invention, various modifications, alternations or changes, made according to the common technical knowledge and conventional means in the art, can also be realized.

### DESCRIPTION OF FIGURES

FIG. 1 The gross appearance of polyurethane microsphere. Polyurethane microspheres are presented as white uniform spherical shapes, and the particle diameter ranges from 150 μm to 270 μm;

FIG. 2 The surface morphology of polyurethane microsphere. The morphology of polyurethane microsphere is observed by scanning electron microscope, and the polyurethane microspheres are presented as spherical shapes, and the surface is smooth;

FIG. 3 The NMR analysis of polyurethane microsphere. Using CHCl₃ as solvent, polyurethane microspheres are dissolved, and ¹H-NMR spectrum is measured, in which 4.1 ppm is ascribe to polycaprolactone, while 3.7 ppm is assigned to polyethylene glycol;

FIG. 4 FTIR analysis of polyurethane microsphere. The absorption bands at 3250-3500 cm⁻¹ are the stretching vibration of —OH and NH of IPDI; the stretching vibration absorption bands of ester group C═O C═O appear at about 1740 cm⁻¹; the absorption band at 1520-1560 cm⁻¹ is the deformation vibration of amide bond N—H. There is no absorption band at 2270 cm⁻¹ indicating NCO of IPDI completely reacted;

FIG. 5 Cell viability on the surface of polyurethane microcarrier and commercial available CultiSpher G microcarrier. Using the cells cultured in plate culture (TCP) and commercial available microcarrier (Cultispher G) as control, the absorbance of cells in the same cultivation volume is measured at different time points using CCK-8, and the result proves the non-toxicity of polyurethane microsphere, and it can effectively promote the expansion of cells in short time;

FIG. 6 Cell distribution on the surface of polyurethane microsphere (7 d)., cells are seeded on microcarrier and subjected to the suspension culture. After 7 days, cells are dyed via DAPI, and the cell nucleus reacts with the staining solution, thus cells present blue under fluorescence excitation. Cells are observed uniformly distributing on the surface of carrier by laser confocal microscopy, indicating the material has good cell compatibility;

FIG. 7 The picture of injectability of polyurethane microsphere;

FIG. 8 The picture of injectability of polyurethane microsphere.

### EMBODIMENT

Main material, reagent, and apparatus:

| Reagent | Abbreviation | Grade | Manufacturer |
|---|---|---|---|
| polycaprolactone 1000 (i.e. | PCL1000 | | Aldrich |
| poly(caprolactone)diol1000) | | | |
| polyethylene glycol 200 | PEG200 | | Meilun Biotech |
| isophorone diisocyanate | IPDI | 99% | Aladdin |
| 2,2-dihydroxymethylbutyric | DMPA | 98% | Aladdin |
| acid | | | |
| triethylamine | TEA | 99% | Kemiou |

oligodiols (polyethylene glycol, poly(caprolactone)diol1000, polytetrahydrofuran);
isocyanate (isophorone diisocyanate, L-lysine diisocyanate, diphenylmethane diisocyanate);
chain extender (2,2-dihydroxymethylbutyric acid, 2,2-dihydroxymethylpropionic acid);
triethylamine, cells (osteoblasts, fibroblasts or stem cells);
PBS without Ca²⁺ and Mg²⁺.
Apparatus: CELLSPIN revolving bottle and double-shaft rotating reactor (INTEGRABiosciences AG), enhanced electric agitator (Jiangsu Jintan Jiamei Instrument).

### Example 1 Preparation of Polyurethane Microsphere According to the Present Invention

1. Preparation

The preparation method of polyurethane microsphere carrying cells includes the following steps:

(1) Oligodiols Premix

Poly(caprolactone)diol 1000 and PEG200 at a molar ratio of 1:1 were added to a three-neck flask, and mixed under stirring at 70° C.;

(2) Pre-Polymerization:

Isophorone diisocyanate and diols in step (1) are starting materials, and added to the reactor, then stirred at the speed of 300 rpm and reacted 2 h;

The molar ratio of isophorone isocyanate and total oligodiols was 2:1;

(3) Chain-Extension Reaction

After above-mentioned step (2), 2,2-dihydroxymethylpropionic acid was added, and the temperature was simultaneously reduced to 45° C., and the mixture was stirred at the speed of 700 rpm to react 2 h;

Wherein, the molar ratio of the chain extender and isocyanate in step (2) was 0.1:1;

(4) Neutralization:

Neutralizer triethylamine was added, and the mixture was allowed to continually react 15 min at the stirring speed of 300 rpm;

Wherein, the neutralizer and the chain extender in step (3) are equimolar;

(5) Emulsification:

The synthesized polyurethane was added dropwise to the distilled water under stirring and dispersed, in which the stirring speed was 700 rpm;

(6) Purification, Sieving and Collection

The polyurethane particles obtained by reaction of step (5) are repeatedly washed with the distilled water (ultrasonic cleaning at room temperature, more than 3 times, each time for 10 minutes), dried in vacuum at room temperature to the constant weight, and sieved with 50-100 meshes to select the microspheres with particle diameter of 150-270 μm.

### Example 2 Preparation of Polyurethane Microsphere According to the Present Invention

1. Preparation

The preparation method of polyurethane microsphere carrying cells includes the following steps:

(1) Oligodiols Premix

Polytetrahydrofuran and PEG200 at a molar ratio of 1.5:1 were added to a three-neck flask, and mixed under stirring at 70° C.;

(2) Pre-Polymerization:

Isophorone diisocyanate and diols in step (1) are starting materials, and added to the reactor, then stirred at the speed of 700 rpm and reacted 3 h;

The molar ratio of isophorone isocyanate and total oligodiols was 2.5:1;

(3) Chain-Extension:

After above-mentioned step (2), 2,2-dihydroxymethylpropionic acid was added, and the temperature was simultaneously reduced to 50° C., and the mixture was stirred at the speed of 300 rpm to react 3 h;

Wherein, the molar ratio of the chain extender and isocyanate in step (2) was 1:1;

(4) Neutralization:

Neutralizer triethylamine was added, and the mixture was allowed to continually react 15 min at the stirring speed of 700 rpm;

Wherein, the neutralizer and the chain extender in step (3) are equimolar;

(5) Emulsification:

The synthesized polyurethane was added dropwise to the distilled water under stirring and dispersed, in which the stirring speed was 300 rpm;

(6) Purification, Sieving and Collection

The polyurethane particles obtained by reaction of step (5) are repeatedly washed with the distilled water (ultrasonic cleaning at room temperature, more than 3 times, each time for 10 minutes), dried in vacuum at room temperature to the constant weight, and sieved with 50-100 meshes to select the microspheres with particle diameter of 150-270 μm.

### Example 3 Preparation of Polyurethane Microsphere According to the Present Invention

1. Preparation

The preparation method of polyurethane microsphere carrying cells includes the following steps:

(1) Oligodiols Premix

Poly(caprolactone)diol 1000 and PEG200 at a molar ratio of 2:1 were added to a three-neck flask, and mixed under stirring at 70° C.;

(2) Pre-Polymerization:

Isophorone diisocyanate and diols in step (1) are starting materials, and added to the reactor, then stirred at the speed of 380 rpm and reacted 4 h;

The molar ratio of isophorone isocyanate and total oligodiols was 2.5:1;

(3) Chain-Extension:

After above-mentioned step (2), 2,2-dihydroxymethylpropionic acid was added, and the temperature was simultaneously reduced to 55° C., and the mixture was stirred at the speed of 380 rpm to react 2 h;

Wherein, the molar ratio of the chain extender and isocyanate in step (2) was 1:1;

(4) Neutralization:

Neutralizer triethylamine was added, and the mixture was allowed to continually react 15 min at the stirring speed of 380 rpm;

Wherein, the neutralizer and the chain extender in step (3) are equimolar;

(5) Emulsification:

The synthesized polyurethane was added dropwise to the distilled water under stirring and dispersed, in which the stirring speed was 500 rpm;

(6) Purification, Sieving and Collection

The polyurethane particles obtained by reaction of step (5) are repeatedly washed with the distilled water (ultrasonic cleaning at room temperature, more than 3 times, each time for 10 minutes), dried in vacuum at room temperature to the constant weight, and sieved with 50-100 meshes to select the microspheres with particle diameter of 150-270 μm.

### Example 4 Preparation of Polyurethane Microsphere According to the Present Invention

1. Preparation

The preparation method of polyurethane microsphere carrying cells includes the following steps:

(1) Oligodiols Premix

Poly(caprolactone)diol 1000 and PEG200 at a molar ratio of 2:1 were added to a three-neck flask, and mixed under stirring at 70° C.;

(2) Pre-Polymerization:

Isophorone isocyanate and diols in step (1) are starting materials, and added to the reactor, then stirred at the speed of 380 rpm and reacted 2.5 h;

The molar ratio of isophorone isocyanate and total oligodiols was 3:1;

(3) Chain-Extension:

After above-mentioned step (2), 2,2-dihydroxymethylbutyric acid was added, and the temperature was simultaneously reduced to 50° C., and the mixture was stirred at the speed of 380 rpm to react 1.5 h;

Wherein, the molar ratio of the chain extender and isocyanate in step (2) was 0.5:1;

(4) Neutralization:

Neutralizer triethylamine was added, and the mixture was allowed to continually react 15 min at the stirring speed of 380 rpm;

Wherein, the neutralizer and the chain extender in step (3) are equimolar;

(5) Emulsification:

The synthesized polyurethane was added dropwise to the distilled water under stirring and dispersed, in which the stirring speed was 500 rpm;

(6) Purification, Sieving and Collection

The polyurethane particles obtained by reaction of step (5) are repeatedly washed with the distilled water (ultrasonic cleaning at room temperature, more than 3 times, each time for 10 minutes), dried in vacuum at room temperature to the constant weight, and sieved with 50-100 meshes to select the microspheres with particle diameter of 150-270

2. Property

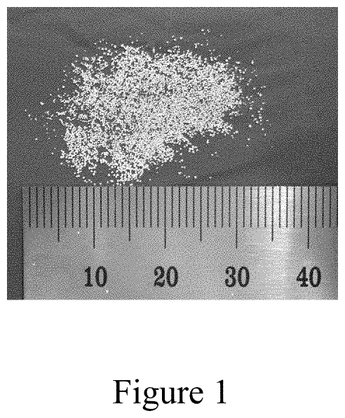

As shown in FIG. 1, polyurethane microspheres prepared as the method of the present invention are presented as white uniform round shapes, and the particle diameter ranges from 150 μm to 270 μm:

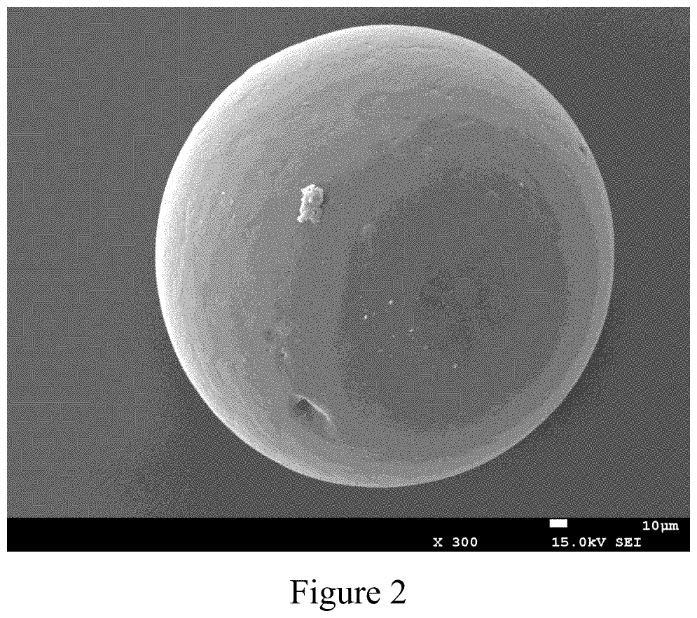

As shown in FIG. 2, the appearance of polyurethane microsphere prepared as the method of the present invention is observed using scanning electron microscope, and microspheres are presented as round shapes, and the surface is smooth and glossy;

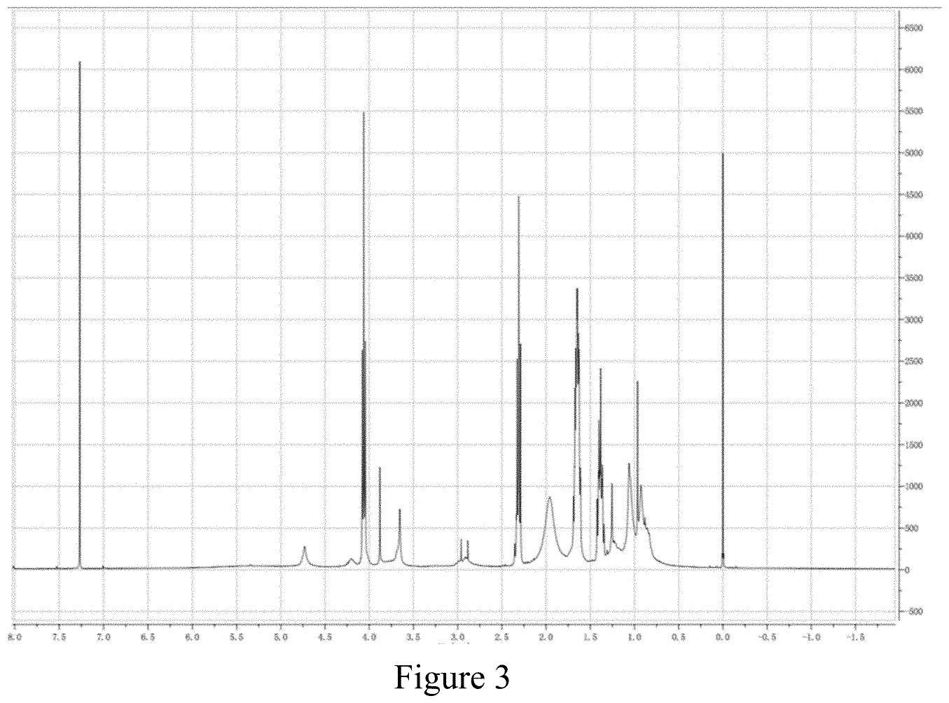

As shown in FIG. 3, using CHCl₃ as solvent, polyurethane microspheres are dissolved, and ¹H-NMR spectrum is measured, in which 4.1 ppm is ascribe polycaprolactone, while 3.7 ppm from polyethylene glycol;

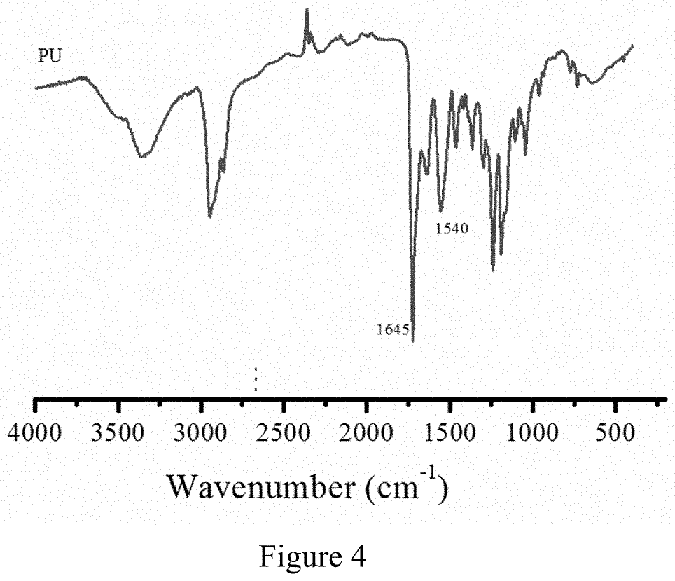

As shown in FIG. 4, the absorption bands at 3250-3500 cm⁻¹ are the stretching vibration of —OH and NH of NHCO in IPDI; the stretching vibration absorption bands of ester group C═O and amide bond C═O appear at about 1740 cm⁻¹; the absorption band at 1520-1560 cm⁻¹ is the deformation vibration of amide bond N—H. There is no absorption band at 2270 cm⁻¹ belonging to NCO of IPDI completely reacted.

### Example 5 Preparation of Polyurethane Microsphere According to the Present Invention

1. Preparation

The preparation method of polyurethane microsphere carrying cells includes the following steps:

(1) Oligodiols Premix

Poly(caprolactone)diol 1000 and PEG200 at a molar ratio of 1:1 were added to a three-neck flask, and mixed under stirring at 70° C.;

(2) Pre-Polymerization:

Isophorone isocyanate and diols in step (1) are starting materials, and added to the reactor, then stirred at the speed of 400 rpm and reacted 3.5 h;

The molar ratio of isophorone isocyanate and total oligodiols was 3:1;

(3) Chain-Extension:

After above-mentioned step (2), 2,2-dihydroxymethylpropionic acid was added, and the temperature was simultaneously reduced to 50° C., and the mixture was stirred at the speed of 400 rpm to react 1 h;

Wherein, the molar ratio of the chain extender and isocyanate in step (2) was 1:1;

(4) Neutralization:

Neutralizer triethylamine was added, and the mixture was allowed to continually react 15 min at the stirring speed of 400 rpm;

Wherein, the neutralizer and the chain extender in step (3) are equimolar;

(5) Emulsification:

The synthesized polyurethane was added dropwise to the distilled water under stirring and dispersed, in which the stirring speed was 600 rpm;

(6) Purification, Sieving and Collection

The polyurethane particles obtained by reaction of step (5) are repeatedly washed with the distilled water (ultrasonic cleaning at room temperature, more than 3 times, each time for 10 minutes), dried in vacuum at room temperature to the constant weight, and sieved with 50-100 meshes to select the microspheres with particle diameter of 150-270 μm.

In the following, the beneficial effect of the present invention is confirmed by example:

### Example 1 Performance Test of Polyurethane Microsphere According to the Present Invention

Polyurethane microspheres prepared in example 4 were adopted, to test their following performances:

### I. Experimental Method

### 1. Cell Expansion Property and Cell Compatibility

Cells were cultured on the surface of polyurethane microcarrier that was realized by the following method:

### (1) Sterilization and Hydration of Microcarrier Material According to the Present Invention

Dried microcarrier (polyurethane microsphere prepared in example 4 of the present invention) (50 mg) was irradiated under UV for 6 h, and added to the silicified glass bottle, then mixed with 10 ml phosphate-buffered saline without Ca²⁺ and Mg²⁺ at room temperature;

### (2) Seeding Cells

Microcarriers in step (1) were centrifugated, and mixed with 50 ml cell medium, and then the mixture was added to the double-shaft rotating reactor, to which was added 5×10⁶ of fibroblasts suspension (1 ml).

The cells cultured on commercial available microcarrier (Cultispher G) and plate culture were used as control group, and other conditions were same to those of microcarrier according to the present invention.

### (3) Cell Expansion

The rotatory speed of reactor was set as 40 rpm, and the bio-reactor was placed at 5% CO₂/37° C.

### (4) Detection

The absorbance of cells was detected at 3 h, 1 d, 3 d, and 7 d of cultivation.

After cultivating for 7 days, cell compatibility was detected, i.e. cells were dyed via DAPI, and the cell nucleus reacted with the staining solution, thus cells presented blue under fluorescence excitation.

### 2. Injectability

Dried microcarrier (polyurethane microsphere prepared in example 4 of the present invention) (50 mg) was irradiated under UV for 6 h, and added to the silicified glass bottle, then hydrated with 10 ml PBS without Ca′ and Mg′ at room temperature, and suctioned with syringe to detect whether the microcarrier is injectable.

### II. Experimental Results

### 1. Property of Amplify Cells

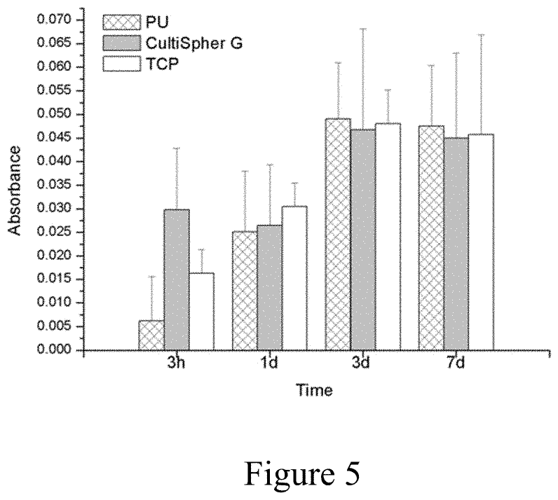

Performance of amplifying cells is shown in FIG. 5. Using the cells cultured on plate culture (TCP) and commercial available microcarrier (Cultispher G) as control, the absorbance of cells in the same cultivation volume was measured at different time points using CCK-8, and the result proved the polyurethane microsphere according to the present invention could effectively promote the expansion of cells in short time, and the effect was obviously better than commercially available gelatin microcarrier.

### 2. Cell Compatibility

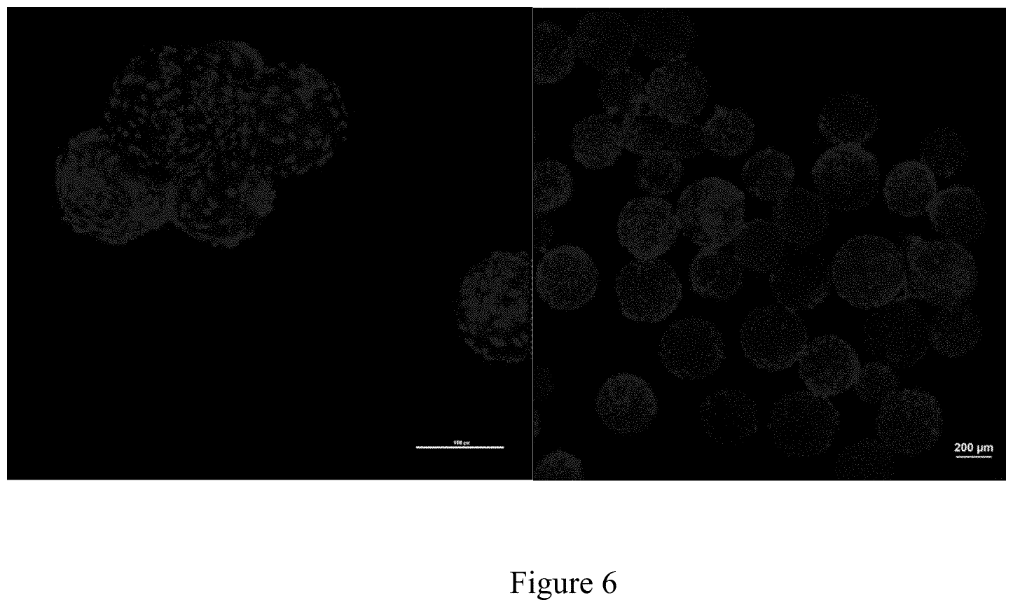

As shown in FIG. 6, after seeding on polyurethane microsphere of the present invention, cells were subjected to the suspension culture system for 7 days. Cells were dyed via DAPI, and the cell nucleus reacted with the staining solution, thus cells present blue under fluorescence excitation. Cells were observed under laser confocal microscopy, and cells uniformly distributed on the surface of microcarrier, indicating the material was non-toxic, and had good cell compatibility.

### 3. Injectability

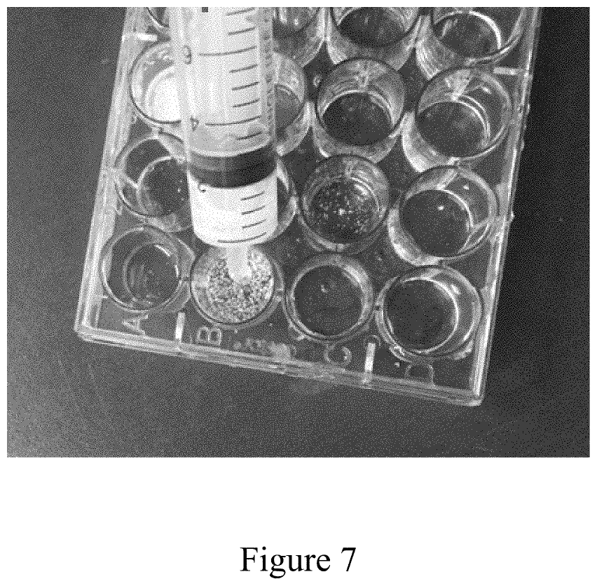

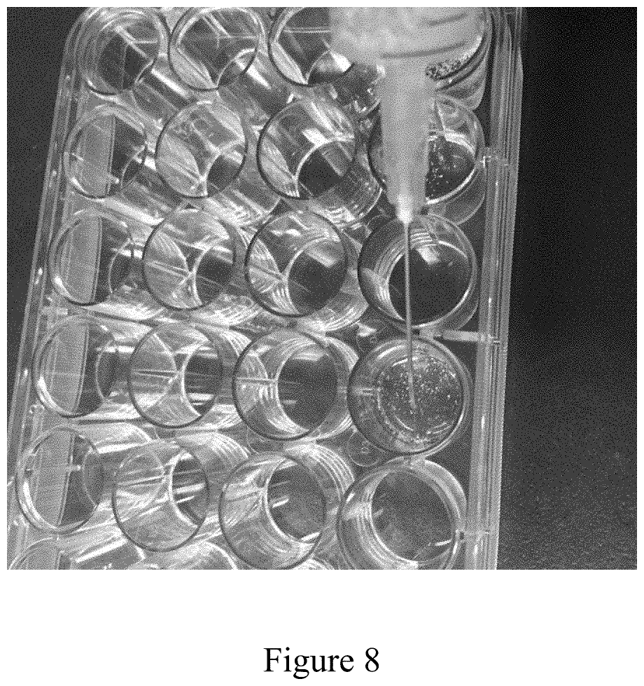

As shown in FIGS. 7 and 8, the polyurethane microsphere according to the present invention can pass through the syringe and its pinhead, demonstrating it is injectable, and be able to easily use in the tissue repair.

To sum up, the polyurethane microsphere which is prepared by this method has good biocompatibility, and it can be used as microcarrier and enhances cell proliferation. Meanwhile, the polyurethane microsphere is injectable and enables to be used in tissue repair with the advantages of good effect, safety and convenience, evidently showing a well clinical application prospect.

1. A polyurethane microsphere, characterized in that its particle diameter is 150 μm-270 μm.

2. The polyurethane microsphere according to claim 1, characterized in that it is prepared as the following method:
(1) Two kinds of different oligodiols are premixed;
(2) Pre-polymerization:
Isocyanate and oligodiols in step (1) are starting materials, and they are added to the reaction vessel and stirred;
(3) Chain-extension:
After above step (2), hydrophilic chain extender is added, and at the same time, the temperature is reduced, and the reactant is stirred;
(4) Neutralization:
Neutralizer is added, and the reactant is continually stirred;
(5) Emulsification:
Under stirring, the polyurethane synthesized in step (4) is added dropwise to the distilled water and dispersed;
(6) The polyurethane microsphere with particle diameter of 150 μm-270 μm is purified, sieved, and collected.

3. The method of polyurethane microsphere according to claim 1, characterized in that the steps are as follows:
(1) Two kinds of different oligodiols are premixed;
(2) Pre-polymerization:
Isocyanate and oligodiols in step (1) are starting materials, and they are added to the reaction vessel and stirred;
(3) Chain-extension:
After above step (2), hydrophilic chain extender is added, and at the same time, the temperature is reduced, and the reactant is stirred;
(4) Neutralization:
After addition of neutralizer, the reactant is continually stirred;
(5) Emulsification:
Under stirring, the polyurethane synthesized in step (4) is added dropwise to the distilled water and dispersed;
(6) The polyurethane microsphere with particle diameter of 150 μm-270 μm is purified, sieved, and collected.

4. The method according to claim 3, characterized in that:
In step (1), two kinds of different oligodiols used in step (1) are optionally selected from the group of polyethylene glycol, poly(caprolactone)diol, and polytetrahydrofuran; preferably, two kinds of different oligodiols used in step (1) are polyethylene glycol and poly(caprolactone)diol or polytetrahydrofuran;
Further, said poly(caprolactone)diol is poly(caprolactone)diol2000 and/or said polyethylene glycol is polyethylene glycol200;
Further, in step (1), the molar ratio of poly(caprolactone)diol and polyethylene glycol is 1:1-2:1;
Further, in step (1), the molar ratio of polytetrahydrofuran and polyethylene glycol is 1:1-2:1;
and/or in step (1), said stirring means mixing at 70° C.

5. The method according to claim 3, characterized in that:
In step (2), the molar ratio of isocyanate and the total oligodiols is (2-3):1, preferably 3:1;
and/or in step (2), said isocyanate is optionally selected from the group of isophorone diisocyanate, L-lysine diisocyanate, and diphenylmethane diisocyanate; preferably, said isocyanate is isophorone diisocyanate;
and/or in step (2), for stirring at speed of 350-700 rpm, preferably, the stirring speed is 380 rpm;
the reaction time is 2-4 hours, preferably 2.5 h.

6. The method according to claim 3, characterized in that in step (3), the molar ratio of said chain extender and isocyanate in step (2) is (0.1-1):(1), preferably 0.5:1;
and/or in step (3), said chain extender is 2,2-dihydroxymethylbutyric acid or 2,2-dihydroxymethylpropionic acid; preferably, said chain extender is 2,2-dihydroxymethylbutyric acid;
and/or in step (3), said reducing the temperature means the temperature is reduced to 45-55° C., preferably 50° C.; said stirring is carried out at the speed of 350-700 rpm, and preferably the stirring speed is 380 rpm; the reaction time is 1-3 hours, preferably 1.5 h.

7. The method according to claim 3, characterized in that in step (4), the neutralizer and the chain extender in step (3) are equimolar;
and/or in step (4), said neutralizer is triethylamine or sodium hydroxide;
and/or in step (4), said stirring is carried out at the speed of 350-700 rpm, preferably the stirring speed is 380 rpm; the reaction time is 15 min.

8. The method according to claim 3, characterized in that in step (5), said stirring speed is 350-700 rpm, preferably 500 rpm.

9. The method according to claim 3, characterized in that the method in step (6) is: the polyurethane particles obtained by reaction of step (5) are washed with the distilled water, dried in vacuum to the constant weight, and sieved with 50-100 meshes to select the microspheres with particle diameter of 150-270 μm.

10. The use of polyurethane microsphere according to claim 1 in the preparation of microcarrier materials.

11. A material for tissue repair in vivo, characterized in that which is prepared by combining the polyurethane microsphere according to claim 1 as microcarrier with cells.
